(19) United States
(12) Patent Application Publication
(10) Pub. No.: US 2025/0049655 A1
(43) Pub. Date: Feb. 13, 2025

(54) OIL-IN-WATER EMULSIFIED COSMETIC COMPOSITION

(71) Applicant: L V M H RECHERCHE, Saint-Jean De Braye (FR)

(72) Inventors: Miyako KITAMURA, Chiyoda-ku, Tokyo (JP); Mai OZAWA, Chiyoda-ku, Tokyo (JP)

(73) Assignee: L V M H RECHERCHE, Saint-Jean De Braye (FR)

(21) Appl. No.: 18/722,739

(22) PCT Filed: Dec. 23, 2021
(86) PCT No.: PCT/IB2021/000905

§ 371 Date: Jun. 21, 2024

(51) Int. Cl. A61K 8/06; A61K 8/44; A61K 8/63; A61K 8/92; A61Q 1/00; A61Q 19/00

(52) U.S. Cl. CPC A61K 8/062; A61K 8/44; A61K 8/63; A61K 8/922; A61Q 1/00; A61Q 19/00

(57) ABSTRACT

The invention relates to an oil-in-water emulsified cosmetic composition comprising (A) hydrogenated lecithin, (B) a phytosterol, (C) an acylglutamic acid with 12 to 22 carbon atoms in an acyl group, or a salt thereof, (D) an oil agent including at least (D1) a vegetable oil, and (E) an aqueous medium.

### TECHNICAL FIELD

The present invention relates to an oil-in-water emulsified cosmetic composition.

### BACKGROUND ART

Oil-in-water emulsions are a form of cosmetics products providing a nourishing effect and protective effect by the oils they contain. Oil-in-water emulsions with high viscosity are used in skin care products such as cosmetic creams and essences.

An example of a skin care product is disclosed in Japanese Unexamined Patent Publication No. 2017-105766, as a gelatinous oil-in-water emulsified composition that can be emulsified under high pressure conditions of 100 MPa and higher. The publication states that emulsification is preferably carried out under high pressure conditions from the viewpoint of the transparency and gel stability over time of the emulsified composition that is obtained.

### Technical Problem

With ever increasing focus on cosmetic safety and preservation of the environment in recent years, demand is increasing for cosmetics with higher contents of naturally-derived components. The oil agents such as hydrocarbon oils and silicone oils that are used in the emulsion of Japanese Unexamined Patent Publication No. 2017-105766 can be switched to naturally-derived oil agents and subjected to emulsification under the high pressure conditions described in the same publication to produce oil-in-water emulsified compositions, but it has been found that their emulsion stability, which is a major important property of such products, is insufficient even when the emulsions are produced using the formulations and methods described therein.

Texture during application is also important for oil-in-water emulsified cosmetics, but oil-in-water emulsified compositions with the oil agents of Japanese Unexamined Patent Publication No. 2017-105766 switched to naturally-derived oil agents are problematic not only because of low stability but also because they cannot be provided with a light texture during application.

Therefore, there is still a need to provide an oil-in-water emulsified cosmetic composition that imparts a light texture during application and emulsion stability (stability allowing the emulsion form to be maintained), even with high contents of naturally-derived components.

### Solution to Problem

In this context, the present invention provides an oil-in-water emulsified cosmetic composition comprising (A) hydrogenated lecithin, (B) a phytosterol, (C) an acylglutamic acid with 12 to 22 carbon atoms in an acyl group, or a salt thereof, (D) an oil agent comprising at least (D1) a vegetable oil, and (E) an aqueous medium. As shown in the examples, said composition imparts a light texture during application and emulsion stability, even with high contents of naturally-derived components.

The “(A) hydrogenated lecithin” will also be referred to herein as “component (A)”, and the other components will be referred to similarly.

With the aim of obtaining an oil-in-water emulsified cosmetic composition that imparts a light texture even with large amounts of oils, the present inventors have produced an oil-in-water nanoemulsion (O/W nanoemulsion) containing hydrogenated lecithin and sodium surfactin, using a high-pressure emulsifier. However, creaming and coalescence were confirmed in this oil-in-water emulsified cosmetic composition, leading to problems in terms of emulsion stability. Substitutes for improving the emulsion stability were therefore examined, and it was found that in order to prevent aggregation it is necessary to modify the three-dimensional conformation of the surfactant at the interface, upon which it was further found that it is useful to use amino acid-based surfactants (component (C) mentioned above) which are less bulky than sodium surfactin.

Although the emulsion stability was improved by using amino acid-based surfactants, coalescence was observed when vegetable oils were used as oil agents to increase the naturally-derived component content. This was attributed to the fact that vegetable oils have high IOB (inorganic-organic balance) values and therefore have high affinity for the lipophilic groups of surfactants, thereby resulting in collapse of the conformation. Upon further examining this problem, it was found that a new emulsified system combining hydrogenated lecithin, phytosterol and an amino acid-based surfactant can remarkably lighten the texture during application while also notably increasing the emulsion stability, even when using vegetable oils as oil agents. This performance is due to a synergistic effect of the constituent components in the emulsified system, and it is not possible to obtain such an excellent effect when even any one of the constituent components of the emulsified system is lacking.

In a particular embodiment, component (A) and component (B) form a complex in the oil-in-water emulsified cosmetic composition. By using a complex of component (A) and component (B), the solubility and dispersibility in aqueous solvents is more superior than with either component (A) or component (B) alone, and collapse of the conformation due to the high IOB value of the vegetable oil can be inhibited, resulting in greater stabilization of the emulsion.

In a particular embodiment, component (C) in the oil-in-water emulsified cosmetic composition comprises stearoylglutamic acid or a salt thereof.

In a particular embodiment, component (D) in the oil-in-water emulsified cosmetic composition further comprises (D2) a non-vegetable polar oil, and the mass ratio (R1) of the component (D2) to the component (D1) (R1=D2/D1) preferably ranges from 0.01 to 1.

In a particular embodiment, component (D1) in the oil-in-water emulsified cosmetic composition is selected from the group consisting of: unsaturated hydrocarbon-based plant oils, and in particular unsaturated fatty acid triglycerides such as meadowfoam seed oil, soybean oil, castor oil, olive oil, ximenia oil, pracaxi oil, palm oil, linseed oil, sunflower oil, argan oil, rice bran oil, avocado oil, macadamia oil, peach kernel oil, apricot kernel oil, rapeseed oil, sweet almond oil, canola oil, coriander oil, cottonseed oil, sesame oil, corn germ oil, wheat germ oil, passionflower oil, grapeseed oil, safflower oil, evening-primrose oil, walnut oil, blackcurrant pip oil, musk rose oil, camellina oil, raspberry seed oil, cranberry oil, castor oil, meadowfoam oil, annatto oil, andiroba oil, Brazil nut oil, buriti oil, mongongo oil, marula oil, coffee oil, Inca inchi oil, jojoba oil, shea butter oil, and mixtures thereof.

In a particular embodiment, component (D2) in the oil-in-water emulsified cosmetic composition is selected from the group consisting of: fatty alcohols that are liquid at ambient temperature, with a branched and/or unsaturated carbon chain having from 18 to 20 carbon atoms, triglycerides of glycerol and C14-C22 fatty acids, dialkylcarbonates, such as dicaprylyl carbonate, and mixtures thereof.

In a particular embodiment, the oil-in-water emulsified cosmetic composition further comprises a hydrophilic thickener. Advantageously, even when a hydrophilic thickener is combined to form a cosmetic cream, a light texture during application is obtained and the emulsion stability is excellent. In particular, water-soluble polymers may be mentioned as thickeners.

In a particular embodiment, the hydrophilic thickener in the oil-in-water emulsified cosmetic composition is selected from the group consisting of: acrylates/C10-30 alkyl acrylates, acrylates/C10-30 alkyl acrylate crosspolymer, hydroxyethyl acrylate/acryloyldimethyltaurine sodium copolymer and mixtures thereof, preferably 2-acrylamido 2-methylpropane sulfonic acid (AMPS) copolymers, acid acrylic/C10-C30 alkylacrylate copolymers and mixtures thereof.

In a particular embodiment, the total content of component (A) and component (B) in the oil-in-water emulsified cosmetic composition ranges from 0.01 to 10 mass %, preferably from 0.05 to 5 mass %, more preferably from 0.1 to 2.5 mass %, based on the total mass of the oil-in-water emulsified cosmetic composition. In a particular embodiment, the total content of component (C) ranges from 0.01 to 5 mass %, preferably from 0.05 to 2 mass %, more preferably from 0.05 to 1 mass %, based on the total mass of the oil-in-water emulsified cosmetic composition.

In a particular embodiment, the total content of the component (D) in the oil-in-water emulsified cosmetic composition ranges from 5 to 60 mass %, preferably from 5 to 50 mass %, more preferably from 10 to 25 mass %, based on the total mass of the oil-in-water emulsified cosmetic composition.

In a particular embodiment, the oil-in-water emulsified cosmetic composition further contains at least a polyol, preferably at least two polyols, preferably selected from the group consisting of: glycerin, propylene glycol, butylene glycol, pentylene glycol and mixtures thereof.

In a particular embodiment, the oil-in-water emulsified cosmetic composition is a nanoemulsion.

In a particular embodiment, the mean particle size of the oil-in-water emulsified cosmetic composition is 50 to 500 nm, for example, as measured by the dynamic light scattering method.

The present invention also provides a cosmetic process for caring for and/or making-up keratinic materials, comprising the application onto keratinic materials, in particular onto skin, of the aforementioned oil-in-water emulsified cosmetic composition.

The present invention further provides the cosmetic process above, wherein the oil-in-water emulsified cosmetic composition exhibits long-term emulsion stability and provides to the keratinic materials on which it is applied, a light texture and richness with a moisturizing effect.

### Advantageous Effects of Invention

According to the invention it is possible to provide an oil-in-water emulsified cosmetic composition that imparts a light texture during application and has excellent emulsion stability, even with an increased content of naturally-derived components. The oil-in-water emulsified cosmetic composition also imparts richness with a moisturizing effect after application.

### DETAILED DESCRIPTION OF THE INVENTION

Preferred embodiments of the invention will now be described in detail. However, the present invention is not limited to the embodiments described below.

The oil-in-water emulsified cosmetic composition according to the invention comprises (A) hydrogenated lecithin, (B) a phytosterol, (C) an acylglutamic acid with 12 to 22 carbon atoms in an acyl group, or a salt thereof, (D) an oil agent including at least (D1) a vegetable oil, and (E) an aqueous medium.

### Component (A)

The oil-in-water emulsified cosmetic composition comprises hydrogenated lecithin as component (A). Hydrogenated lecithin has hydrogen added to lecithin (by hydrogenation). Lecithin has hydrophobic groups and hydrophilic groups, the hydrophobic groups being two acyl groups (fatty acid residues), and the hydrophilic groups being the anion group of the phosphoric acid ester salt and the cation group of the quaternary ammonium salt. Fatty acid residues in lecithin derived from egg yolk are said to be fatty acid residues comprising palmitic acid, stearic acid, oleic acid, linoleic acid, linolenic acid and arachidic acid, and those derived from soybean are said to be fatty acid residues comprising palmitic acid, stearic acid, oleic acid, linoleic acid and linolenic acid. Hydrogenated lecithin obtain by hydrogenation of such lecithin has a structure in which the hydrophobic groups are saturated while the hydrophilic groups are maintained. In other words, hydrophobic fatty acid residues are considered to be palmitic acid, stearic acid, arachidic acid and behenic acid residues in the case of egg yolk lecithin, and palmitic acid and stearic acid residues in the case of soybean lecithin.

In the context of the present invention, the hydrogenated lecithin may be a hydrogenated product of natural lecithin extracted from egg yolk, soybean, corn or rapeseed, or a hydrogenated product of synthetic lecithin. From the viewpoint of obtaining a high content of naturally-derived components, the hydrogenated lecithin is preferably a hydrogenated product of natural lecithin. In an embodiment, component (A) consists of a single component as described above. In another embodiment, it is a mixture of two or more of the aforementioned components. When it is a mixture, it may be a mixture of components from different sources, or a mixture of components from the same source but with different degrees of hydrogenation, or a mixture of components from different sources and with different degrees of hydrogenation. In a preferred embodiment, the degree of hydrogenation of the hydrogenated lecithin is essentially 100% (fully hydrogenated lecithin).

The total content of component(s) (A) preferably ranges from 0.01 to 10 mass %, preferably from 0.05 to 5 mass %, and still preferably from 0.1 to 2.5 mass %, based on the total mass of the oil-in-water emulsified cosmetic composition.

### Component (B)

The oil-in-water emulsified cosmetic composition comprises a phytosterol as component (B). In an embodiment, a single type of phytosterol is used. In another embodiment, a combination of two or more components (B) are used. In a preferred embodiment, said, one or more phytosterol(s) is selected from the group consisting of: among β-sitosterol, campesterol, stigmasterol, brassicasterol, and mixtures thereof.

The total content of component (B) preferably ranges from 0.005 to 5 mass %, preferably from 0.01 to 2.5 mass % and more preferably from 0.02 to 1 mass %, based on the total mass of the oil-in-water emulsified cosmetic composition.

The mass ratio of component (B) to component (A) preferably ranges from 0.01 to 1, preferably from 0.01 to 0.5, preferably from 0.05 to 0.5, preferably from 0.1 to 0.3 and more preferably from 0.1 to 0.2.

According to a preferred embodiment, the oil-in-water emulsified cosmetic composition has component (A) and component (B) forming a complex. A “complex” is a form in which component (A) and component (B) are co-present by means of physical or chemical interaction. Such forms include forms in which the hydrophilic groups of component (A) and the hydrophilic groups of component (B), and the lipophilic groups of component (A) and the lipophilic groups of component (B), are regularly arranged in the same orientation. The preferred form is a structure similar to a biological membrane. That is, it is a complex with a double layered structure, in which each layer of the double layer having a phytosterol embedded between hydrogenated lecithin with the hydrophilic groups and hydrophobic groups oriented in the same direction, and the layers being arranged so that the hydrophobic groups of the hydrogenated lecithin are facing each other. A complex of this form can be obtained, for example, by uniformly dissolving component (A) and component (B) in an organic solvent, and then removing the organic solvent to simultaneously precipitate component (A) and component (B).

Advantageously, compared to component (A) alone or component (B) alone, a complex of component (A) and component (B) has more excellent dispersibility in aqueous solvents and tends to have more excellent emulsion stability.

The total content of the complex of component (A) and component (B) preferably ranges from 0.01 to 10 mass %, preferably from 0.05 to 5 mass %, and more preferably from 0.1 to 2.5 mass %, based on the total mass of the oil-in-water emulsified cosmetic composition.

### Component (C)

The oil-in-water emulsified cosmetic composition comprises an acylglutamic acid with 12 to 22 carbon atoms in an acyl group, or a salt thereof, as component (C). In an embodiment, component (C) consists of a single component, as mentioned above. In another embodiment, component (C), comprises a mixture of two or more of the aforementioned components. The number of carbon atoms in the acyl group of component (C) preferably ranges from 14 to 20, preferably from 14 to 18, preferably from 16 to 18, and more preferably is 18.

Examples for component (C) include lauroylglutamic acid, myristoylglutamic acid, palmitoylglutamic acid, stearoylglutamic acid and their salts, and mixtures thereof. In a preferred embodiment, component (C) is stearoylglutamic acid or a salt thereof.

Salts of stearoylglutamic acid include sodium salts, potassium salts, calcium salts, aluminum salts and magnesium salts. In a particular embodiment, component (C) includes a sodium salt of an acylglutamic acid, and preferably includes sodium stearoylglutamate.

The total content of stearoylglutamic acid and its salts based on the total mass of component (C) ranges from 50 to 100 mass %, preferably from 80 to 100 mass % and more preferably from 90 to 100 mass %.

The total content of component (C) preferably ranges from 0.01 to 5 mass %, preferably from 0.05 to 2 mass %, more preferably from 0.05 to 1 mass %, based on the total mass of the oil-in-water emulsified cosmetic composition. The total content of stearoylglutamic acid and its salts preferably ranges from 0.01 to 5 mass %, preferably from 0.05 to 2 mass %, and more preferably from 0.05 to 1 mass %, based on the total mass of the oil-in-water emulsified cosmetic composition.

### Component (D)

The oil-in-water emulsified cosmetic composition comprises a vegetable oil (component (D1)) as component (D).

Vegetable oils are oils obtained from plant starting materials. In a particular embodiment, component (D1) is selected from the group consisting of: unsaturated hydrocarbon-based plant oils, and in particular unsaturated fatty acid triglycerides such as meadowfoam seed oil, soybean oil, castor oil, olive oil, ximenia oil, pracaxi oil, palm oil, linseed oil, sunflower oil, argan oil, rice bran oil, avocado oil, macadamia oil, peach kernel oil, apricot kernel oil, rapeseed oil, sweet almond oil, canola oil, coriander oil, cottonseed oil, sesame oil, corn germ oil, wheat germ oil, passionflower oil, grapeseed oil, safflower oil, evening-primrose oil, walnut oil, blackcurrant pip oil, musk rose oil, camellina oil, raspberry seed oil, cranberry oil, castor oil, meadowfoam oil, annatto oil, andiroba oil, Brazil nut oil, buriti oil, mongongo oil, marula oil, coffee oil, Inca inchi oil, jojoba oil, shea butter oil, and mixtures thereof. In a particular embodiment, component (D) includes at least meadowfoam seed oil.

The total content of component (D1) preferably ranges from 1 to 60 mass %, preferably from 5 to 50 mass %, and more preferably from 10 to 25 mass %, based on the total mass of the oil-in-water emulsified cosmetic composition.

In some embodiments, the oil-in-water emulsified cosmetic composition does not comprise a non-vegetable oil as component (D). In some particular embodiments, the oil-in-water emulsified cosmetic composition comprises a non-vegetable oil as component (D). Non-vegetable oils are oils other than vegetable oils, and they include animal oils and mineral oils. A non-vegetable oil may be either a polar non-vegetable oil (component (D2)) or a non-polar non-vegetable oil, but it is preferably a polar non-vegetable oil from the viewpoint of obtaining a light texture.

Preferably, said non-vegetable oil is selected from the group consisting of: monoesters of aliphatic carboxylic acids, monoesters of aliphatic alcohols, dialkyl carbonates and saturated or unsaturated aliphatic alcohols, hydrocarbon oils or silicone oils. Specific non-vegetable oils include ethyl-2-hexyl benzoate, octyl-2-dodecyl benzoate, isostearyl benzoate, pentaerythrityl tetraisostearate, trimethylolpropane triisostearate, tridecyl citrate, tridecyl trimellitate, dicaprylyl carbonate, di(ethyl-2-hexyl)-carbonate, isostearyl lactate, octyl hydroxystearate, octyldodecyl hydroxystearate, cetyl lactate, myristyl lactate, alkanes, polyolefins, hydrogenated polyolefins, squalane, phenyltrimethicone, phenyldimethicone, phenyltrimethylsiloxydiphenylsiloxane, diphenyldimethicone, diphenylmethyldiphenyltrisiloxane,2-phenylethyl trimethylsiloxysilicate, and mixtures thereof. In a particular embodiment, the non-vegetable oil is not a silicone oil.

In a particular embodiment, component (D2) is selected from the group consisting of: fatty alcohols that are liquid at ambient temperature, with a branched and/or unsaturated carbon chain having from 18 to 20 carbon atoms, triglycerides of glycerol and C14-C22 fatty acids, dialkylcarbonates, such as dicaprylyl carbonate and mixtures thereof.

In a particular embodiment, the oil-in-water emulsified cosmetic composition further comprises a perfume oil as component (D). A perfume oil may be one qualifying as component (D1), or one qualifying as component (D2).

The total content of component (D2) preferably ranges from 0.01 to 20 mass % and preferably from 0.05 to 15 mass %, based on the total mass of the oil-in-water emulsified cosmetic composition.

The total content of component (D) preferably ranges from 1 to 60 mass %, preferably from 5 to 50 mass %, and more preferably from 10 to 25 mass %, based on the total mass of the oil-in-water emulsified cosmetic composition.

When the oil-in-water emulsified cosmetic composition comprises component (D1) and component (D2) as component (D), the mass ratio (R1) of component (D2) with respect to component (D1) (R1=D2/D1) ranges from 0.01 to 1, preferably from 0.05 to 1 and more preferably from 0.1 to 1.

### Component (E)

The oil-in-water emulsified cosmetic composition comprises an aqueous medium as component (E). In an embodiment, the aqueous medium is composed entirely of water. In another embodiment, the aqueous medium is composed of water and a water-soluble solvent (for example, a water-soluble low molecular mono-alcohol).

The total content of component (E) preferably ranges from 40 to 90 mass %, from 50 to 80 mass %, and preferably from 55 to 70 mass %, based on the total mass of the oil-in-water emulsified cosmetic composition.

In an embodiment, the oil-in-water emulsified cosmetic composition further comprises a polyol. Examples of polyols include glycols such as 1,3-propanediol, propylene glycol, butylene glycol, pentylene glycol, dipropylene glycol and polyethylene glycol; glycerols such as glycerin, diglycerin and polyglycerin; and sugar alcohols such as sorbitol; and mixtures thereof. In a particular embodiment, the oil-in-water emulsified cosmetic composition further comprises at least a polyol, preferably at least two polyols, preferably chosen from glycerin, propylene glycol, butylene glycol, pentylene glycol and mixtures thereof.

In a particular embodiment, a polyol is a low molecular polyol having a molecular weight of 1000 or lower. The molecular weight of the polyol preferably ranges from 50 to 1000, preferably from 70 to 500 and more preferably from 80 to 300.

The total content of the polyol preferably ranges from 1 to 40 mass %, preferably from 5 to 30 mass %, and more preferably from 10 to 25 mass %, based on the total mass of the oil-in-water emulsified cosmetic composition.

In a particular embodiment, the oil-in-water emulsified cosmetic composition further comprises a hydrophilic thickener. The hydrophilic thickener may be a water-soluble polymer, a (meth)acrylic polymer or a polysaccharide. Advantageously, an oil-in-water emulsified cosmetic composition that includes such hydrophilic thickeners exhibits excellent stability while maintaining suitable viscosity.

Preferably, said (meth)acrylic polymer is selected on the group consisting of: acrylates/C10-30 alkyl acrylates, acrylates/C10-30 alkyl acrylate crosspolymer, hydroxyethyl acrylate/acryloyldimethyltaurine sodium copolymer and mixtures thereof.

Preferably, said polysaccharide is selected on the group consisting of: xanthan gum, welan gum, gellan gum, diutan gum, and mixtures thereof.

The total content of the hydrophilic thickener preferably ranges from 0.01 to 10 mass %, preferably from 0.05 to 5 mass %, and more preferably from 0.1 to 1 mass %, based on the total mass of the oil-in-water emulsified cosmetic composition.

In a particular embodiment, the oil-in-water emulsified cosmetic composition also comprises additives commonly used in cosmetics, preferably selected from the group consisting of: as stabilizers, humectants, pH adjustors, perfumes, preservatives, active ingredients, antioxidants, anti-UV agents, skin whiteners, pigments, coloring agents, skin conditioning agents and mixtures thereof.

The oil-in-water emulsified cosmetic composition comprises emulsified particles, with at least one of components (A) to (D) forming the emulsified particles. The oil-in-water emulsified cosmetic composition preferably is a nanoemulsion. The mean particle size in said nanoemulsion preferably ranges from 50 to 500 nm, preferably from 50 to 300 nm and more preferably from 50 to 100 nm, for example, as measured by dynamic light scattering. The mean particle size of the emulsion particles can be measured using a particle size measuring device (e.g., DelsaMax CORE: manufactured by Beckman Coulter, Inc.) based on dynamic light scattering.

In an embodiment, the oil-in-water emulsified cosmetic composition according to the invention is produced by stirring and mixing components (A) to (E), with the hydrophilic thickener and stabilizer, while heating at 60 to 85° C., and then cooling the mixture to room temperature and emulsifying it.

In particular, the oil-in-water emulsified cosmetic composition according to the invention can be used in the form of a cream, liquid or cosmetic water. Preferably, the oil-in-water emulsified cosmetic composition according to the invention includes large amounts of naturally-derived components. In particular, the contents of naturally-derived components is preferably 90% or greater, preferably 91% or greater, preferably 92% or greater, preferably 93% or greater, preferably 94% or greater and more preferably 95% or greater, for example, as measured according to IS016128.

Another aspect of the present invention relates to a cosmetic process for caring for and/or making-up keratinic materials comprising the application onto keratinic materials, in particular onto skin, of the oil-in-water emulsified cosmetic composition as described above. In said cosmetic process for caring and/or making-up keratinic materials, the oil-in-water emulsified cosmetic composition exhibits a long emulsion stability and provides to the keratinic materials on which it is applied, a light texture and richness with moisturizing effect.

### The Present Invention Will Now be Further Explained Using the Non-Limitative Following Examples and Comparative Examples.

### EXAMPLES

### Example 1 and Comparative Examples 1 to 5

Phases 1 to 3 shown in Table 1 were heated at 75° C., and phase 1 was dissolved by stirring with a stirrer at 1000 rpm. Phase 2 was added to phase 1 and the mixture was stirred with a stirrer at 1000 rpm for 10 minutes to obtain a mixture of phase 1 and phase 2. Phase 3 was added to the mixture of phase 1 and phase 2, and the resulting mixture was stirred with a stirrer at 1000 rpm for 10 minutes to obtain a mixture of phases 1 to 3. The mixture of phases 1 to 3 was cooled to room temperature and dispersed 3 times at 200 MPa using a Starburst Mini (Sugino Machine Limited). An O/W nano-emulsion was thus obtained.

### Example 2

An O/W nano-emulsion was obtained from phases 1 to 3 in the same manner as Example 1. Phase 4 that had been dispersed by stirring with a stirrer at 1000 rpm was added to the O/W emulsion, and the resulting mixture was stirred with a stirrer at 1000 rpm for 10 minutes. A cream comprising the O/W emulsion was thus obtained.

TABLE 1

| | | Example 1 | Example 2 | Comp. | Comp. | Comp. | Comp. | Comp. |
|---|---|---|---|---|---|---|---|---|
| Phase | | invention | invention | Ex. 1 | Ex. 2 | Ex. 3 | Ex. 4 | Ex. 5 |
| 1 | Complex of | 1 | 1 | — | 0 | 1.2 | 1 | 1 |
| | hydrogenated lecithin | | | | | | | |
| | and phytosterols | | | | | | | |
| | Hydrogenated lecithin | — | — | 1 | — | — | — | — |
| | Sodium stearoyl | 0.2 | 0.2 | 0.2 | 1.2 | 0 | 0 | 0.2 |
| | glutamate | | | | | | | |
| | Sodium surfactin | — | — | — | — | — | 0.2 | — |
| | Glycerin | 10 | 10 | 10 | 10 | 10 | 10 | 10 |
| | Butylene glycol | 10 | 10 | 10 | 10 | 10 | 10 | 10 |
| 2 | Meadowfoam seed oil | 10 | 10 | 10 | 10 | 10 | 10 | — |
| | Dicaprylyl carbonate | 10 | 10 | 10 | 10 | 10 | 10 | 10 |
| | Hydrogenated | — | — | — | — | — | — | 10 |
| | polydecene | | | | | | | |
| | Perfume | 0.1 | 0.1 | 0.1 | 0.1 | 0.1 | 0.1 | 0.1 |
| 3 | Water | 58.2 | 28.2 | 58.2 | 58.2 | 58.2 | 58.2 | 58.2 |
| | Preservatives | 0.5 | 0.5 | 0.5 | 0.5 | 0.5 | 0.5 | 0.5 |
| 4 | Acrylates/C10-30 alkyl | — | 0.2 | — | — | — | — | — |
| | acrylate cross polymer | | | | | | | |
| | Sodium hydroxyethyl | — | 0.1 | — | — | — | — | — |
| | acrylate/acryloyl | | | | | | | |
| | dimethyl | | | | | | | |
| | taurate copolymer | | | | | | | |
| | Sodium hydroxide | — | 0.1 | — | — | — | — | — |
| | Water | — | 29.6 | — | — | — | — | — |

The following are the details far the materials listed in the tables.
Complex of hydrogenated lecithin and phytosterols: Phytocompo PP, Nippon Seika Co., Ltd.
Hydrogenated lecithin: EMULMETIK 100, Lucas Meyer Cosmetics by IFF Inc.
Sodium stearoyl glutamate: AMISOFT HS-11, Ajinomoto Healthy Supply Co., Inc.
Sodium surfactin: KANEKA SURFACTIN, Kaneka Carp.
Meadowfoam seed oil: FANCOR MEADOWFOAM SEED OIL, Elementis Co.
Dicaprylyl carbonate: CETIOL CC, BASF Co.
Hydrogenated polydecene: SILKFLO 364, Vantage Personal Care Co.
Preservatives: Chlorphenesin and Hydroxyacetophenone
Acrylates/C10-30 alkyl acrylate crass polymer: CARBOPOL ULTREZ 21, Lubrizol Co.
Sodium hydroxyethyl acrylate/acryloyl dimethyl taurate copolymer: SIMULGEL NS, Seppic Co.

### Evaluation

### Measurement of Emulsion Diameter

The mean particle size of the emulsified particles was measured by the dynamic light scattering method using a particle size measuring device (DelsaMax CORE, product of Beckman Coulter, Inc.). The results are shown in Table 2.

### Texture Evaluation

Each sample was applied onto skin by an evaluation panel of cosmetic experts and evaluation was conducted based on the following evaluation scale. The results are shown in Table 2.
A: Very light texture
B: Virtually no light texture
C: Absolutely no light texture

### Emulsion Stability

Each sample was filled into a transparent glass container and sealed. The glass container was then placed in a thermostatic bath at 50° C. and allowed to stand for 1 month. The emulsified stability was visually evaluated based on the following evaluation scale. The results are shown in Table 2.
A: No change
B: Slight creaming and/or coalescence
C: Creaming and/or coalescence
   The compositions having ‘A’ notation for each criteria (texture and stability) are acceptable according to the invention.

TABLE 2

| | Example 1 | Example 2 | Comp. | Comp. | Comp. | Comp. | Comp. |
|---|---|---|---|---|---|---|---|
| | (invention) | (invention) | Ex. 1 | Ex. 2 | Ex. 3 | Ex. 4 | Ex. 5 |
| Average particle | 100 | 100 | 100 | 100 | 200 | 100 | 100 |
| size (nm) | | | | | | | |
| Texture | A | A | A | A | B | A | B |
| evaluation | | | | | | | |
| Emulsion stability | A | A | C | C | B | C | A |

The oil-in-water emulsified cosmetic composition according to the invention, obtained in Examples 1 and 2, comprised large amounts of naturally-derived components, which are hydrogenated lecithin, phytosterol, sodium stearoylglutamate and vegetable oils and imparted a light texture during application, while the emulsions exhibited excellent stability without separation between the phases even after elapse of 1 month at 50° C.

With Comparative Examples 1 to 4, however, which do not comprise component A (comparative example 1), component A and B (comparative example 2) or component C (comparative examples 3 and 4) separation was observed between the phases after elapse of 1 month at 50° C. Moreover, with Comparative Example 5 which had the vegetable oil (component D) replaced by a non-vegetable oil, the emulsion exhibited excellent stability but had unacceptable texture during application.

1. An oil-in-water emulsified cosmetic composition comprising:
(A) hydrogenated lecithin,
(B) a phytosterol,
(C) an acylglutamic acid with 12 to 22 carbon atoms in an acyl group, or a salt thereof,
(D) an oil agent comprising at least (D1) a vegetable oil, and
(E) an aqueous medium.

2. The oil-in-water emulsified cosmetic composition according to claim 1, wherein component (A) and component (B) form a complex.

3. The oil-in-water emulsified cosmetic composition according to claim 1, wherein component (C) comprises stearoylglutamic acid or a salt thereof.

4. The oil-in-water emulsified cosmetic composition according to claim 1, wherein the component (D) further comprises (D2) a non-vegetable polar oil.

5. The oil-in-water emulsified cosmetic composition according to claim 1, wherein the component (D1) is selected from the group consisting of: unsaturated hydrocarbon-based plant oils.

6. The oil-in-water emulsified cosmetic composition according to claim 4, wherein the component (D2) is selected from the group consisting of: fatty alcohols that are liquid at ambient temperature, with a branched and/or unsaturated carbon chain having from 18 to 20 carbon atoms, triglycerides of glycerol and 014-022 fatty acids, dialkylcarbonates, and mixtures thereof.

7. The oil-in-water emulsified cosmetic composition according to claim 1, which further comprises a hydrophilic thickener.

8. The oil-in-water emulsified cosmetic composition according to claim 7, wherein the hydrophilic thickener is selected from the group consisting of: acrylates/C10-30 alkyl acrylates, acrylates/C10-30 alkyl acrylate crosspolymer, hydroxyethyl acrylate/acryloyldimethyltaurine sodium copolymer, and mixtures thereof.

9. The oil-in-water emulsified cosmetic composition according to claim 1, wherein the total content of component (A) and component (B) ranges from 0.01 to 10 mass % based on the total mass of the oil-in-water emulsified cosmetic composition.

10. The oil-in-water emulsified cosmetic composition according to claim 1, wherein the total content of component (C) ranges from 0.01 to 5 mass % based on the total mass of the oil-in-water emulsified cosmetic composition.

11. The oil-in-water emulsified cosmetic composition according to claim 1, wherein the total content of the component (D) ranges from 5 to 60 mass % based on the total mass of the oil-in-water emulsified cosmetic composition.

12. The oil-in-water emulsified cosmetic composition according to claim 1, further comprising at least a polyol or at least two polyols.

13. The oil-in-water emulsified cosmetic composition according to claim 1, which is a nanoemulsion.

14. The oil-in-water emulsified cosmetic composition according to claim 1, wherein the mean particle size ranges from 50 nm to 500 nm as measured by the dynamic light scattering method.

15. A cosmetic process for caring for and/or making-up keratinic materials, comprising the application onto keratinic materials of the oil-in-water emulsified cosmetic composition according to claim 1.

16. The cosmetic process according to claim 15, wherein the oil-in-water emulsified cosmetic composition exhibits long-term emulsion stability and provides to the keratinic materials on which it is applied, a light texture and richness with a moisturizing effect.

17. The oil-in-water emulsified cosmetic composition according to claim 4, wherein the mass ratio (R1) of the component (D2) to the component (D1) (R1=D2/D1) ranges from 0.01 to 1.

18. The oil-in-water emulsified cosmetic composition according to claim 5, wherein the component (D1) is selected from the group consisting of unsaturated fatty acid triglycerides.

19. The oil-in-water emulsified cosmetic composition according to claim 8, wherein the hydrophilic thickener is selected from the group consisting of 2-acrylamido 2-methylpropane sulfonic acid (AMPS) copolymers, acid acrylic/C10-C30 alkylacrylate copolymers, and mixtures thereof.

20. The oil-in-water emulsified cosmetic composition according to claim 12, wherein said polyol(s) is(are) selected from the group consisting of glycerin, propylene glycol, butylene glycol, pentylene glycol, and mixtures thereof.
